(19) United States
(12) Patent Application Publication
(10) Pub. No.: US 2025/0281012 A1
(43) Pub. Date: Sep. 11, 2025

(54) AUTOMATIC TOILET BOWL COVERING DEVICE

(71) Applicant: Terri Donato, Parrish, FL (US)

(72) Inventor: Terri Donato, Parrish, FL (US)

(21) Appl. No.: 19/049,131

(22) Filed: Feb. 10, 2025

Related U.S. Application Data
(60) Provisional application No. 63/563,602, filed on Mar. 11, 2024.

(51) Int. Cl. A47K 13/10; A47K 13/30; A61L 2/10; A61L 2/24

(52) U.S. Cl. CPC A47K 13/10; A47K 13/302; A61L 2/10; A61L 2/24; A61L 2202/11; A61L 2202/122; A61L 2202/14

(57) ABSTRACT

An automatic toilet bowl covering device is provided. The device is comprised of an automatic toilet bowl covering device designed to enhance bathroom hygiene by deploying a flexible, multi-layered cover over the toilet bowl opening during flushing to prevent the dispersion of airborne waste particles. The cover comprises hydrophobic, oleophobic, and antimicrobial layers for durability, cleanliness, and odor control, with accordion-style folds and elastic edging ensuring secure deployment and retraction. A motorized mechanism powered by a brushless DC motor smoothly operates the cover along guide tracks, while sensors automate the system based on user movement, lid position, and flushing activity. A UV-C light sterilizes the cover post-use within a storage compartment.

### CROSS-REFERENCE TO RELATED APPLICATION

The present application claims priority to, and the benefit of, U.S. Provisional Application No. 63/563,602, which was filed on Mar. 11, 2024, and is incorporated herein by reference in its entirety.

### FIELD OF THE INVENTION

The present invention relates generally to the field of toilets. More specifically, the present invention relates to an automatic toilet bowl covering device that deploys a multi-layered, flexible or hard cover to contain airborne waste particles during flushing, enhancing hygiene. A motorized system with integrated sensors automates deployment and retraction, while a UV-C light sterilizes the cover within a durable storage compartment for hygienic reuse. Accordingly, the present disclosure makes specific reference thereto. Nonetheless, it is to be appreciated that aspects of the present invention are also equally applicable to other like applications, devices, and methods of manufacture.

### BACKGROUND

The flushing of toilets introduces a significant hygiene challenge due to the release of airborne waste particles and bacteria into the bathroom environment. These particles can settle on surfaces such as toothbrushes, hairbrushes, cosmetics, and other frequently used items, potentially leading to contamination and posing health risks. This phenomenon also results in water splashes that contaminate the toilet seat and surrounding floor, necessitating regular cleaning. Despite the availability of various toilet designs and accessories aimed at minimizing this issue, most fail to adequately address the dispersion of airborne contaminants during flushing. Additionally, these solutions often lack automation or features that simplify maintenance and ensure consistent hygiene. As a result, there is a persistent need for a more comprehensive and user-friendly approach to containing waste particles and maintaining cleanliness in bathrooms.

Therefore, there exists a long-felt need in the art for an automatic toilet bowl covering device that effectively prevents the dispersion of airborne waste particles during flushing. There also exists a long-felt need in the art for an automatic toilet bowl covering device that minimizes water splashes and prevents contamination of surrounding surfaces. Moreover, there exists a long-felt need in the art for an automatic toilet bowl covering device that automates the process of deploying and retracting a protective cover, ensuring consistent hygiene and ease of use.

The subject matter disclosed and claimed herein, in one embodiment thereof, comprises an automatic toilet bowl covering device. The device is comprised of an automatic toilet bowl covering device designed to enhance bathroom hygiene by deploying a flexible or hard, multi-layered cover over the toilet bowl opening during flushing to prevent the dispersion of airborne waste particles. The cover comprises hydrophobic, oleophobic, and antimicrobial layers for durability, cleanliness, and odor control, with accordion-style folds and elastic edging ensuring secure deployment and retraction. A motorized mechanism powered by a brushless DC motor smoothly operates the cover along guide tracks, while sensors automate the system based on user movement, lid position, and flushing activity. A UV-C light sterilizes the cover post-use within a storage compartment.

In this manner, the automatic toilet bowl covering device of the present invention accomplishes all the forgoing objectives by deploying a flexible or hard, multi-layered cover over the toilet bowl opening during flushing. The cover prevents waste particles and splashes from escaping, thereby protecting nearby surfaces and maintaining hygiene. The device integrates advanced features such as a motorized deployment mechanism, weight sensors, and UV-C light sterilization, which ensure automated operation and consistent sanitization of the cover. By combining durability, ease of maintenance, and effective containment, the device offers a practical and innovative solution to enhance bathroom cleanliness and reduce the risk of contamination.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an automatic toilet bowl covering device. The automatic toilet bowl covering device is a motorized device designed to deploy a flexible or hard cover over the toilet bowl opening, preventing the dispersion of airborne waste particles during flushing.

The flexible or hard cover is comprised of an outer layer with hydrophobic and oleophobic coatings to repel liquids and particles, a middle layer of reinforced mesh for tear resistance and pliability, and an inner layer with antimicrobial coating to inhibit bacterial growth and minimize odor. Accordion-style folds in the cover enable smooth retraction into a storage compartment, while elastic edging ensures a secure seal around the bowl opening.

The storage compartment, which is positioned at the rear of the toilet seat, is constructed from durable materials and incorporates a UV-C light for sterilizing the cover between uses. The UV-C light, powered by a battery with an indicator for status monitoring, neutralizes pathogens, ensuring hygienic readiness.

A motorized mechanism controls the deployment and retraction of the cover, utilizing a brushless DC motor connected to guide roller tracks around the toilet bowl perimeter. The cover retracts compactly into the compartment, with the UV-C light continuing to sterilize during the process. The system operates automatically through sensors: a weight sensor detects when the user rises, motion sensors monitor the toilet lid position, and a flush detection sensor triggers retraction after a delay to ensure effective particle containment.

The device enhances bathroom hygiene by containing waste particles, minimizing contamination, and reducing the need for manual cleaning. The durable, multi-layered cover and integrated UV-C sterilization system offer long-term reliability and convenience.

Accordingly, the automatic toilet bowl covering device of the present invention is particularly advantageous as it provides a flexible, multi-layered cover over the toilet bowl opening during flushing. The cover prevents waste particles and splashes from escaping, thereby protecting nearby surfaces and maintaining hygiene. The device integrates advanced features such as a motorized deployment mechanism, weight sensors, and UV-C light sterilization, which ensure automated operation and consistent sanitization of the cover. By combining durability, ease of maintenance, and effective containment, the device offers a practical and innovative solution to enhance bathroom cleanliness and reduce the risk of contamination.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a top view of one potential embodiment of an automatic toilet bowl covering device of the present invention while attached to a toilet and wherein the cover is retracted within the storage compartment in accordance with the disclosed architecture;

FIG. 2 illustrates a side cross-sectional view of a cover of one potential embodiment of an automatic toilet bowl covering device of the present invention in accordance with the disclosed architecture;

FIG. 3 illustrates a top view of one potential embodiment of an automatic toilet bowl covering device of the present invention while attached to a toilet and wherein the cover is extended from the storage compartment while covering the toilet bowl of the toilet in accordance with the disclosed architecture; and

FIG. 4 illustrates a perspective view of one potential embodiment of an automatic toilet bowl covering device of the present invention while attached to a toilet and wherein the cover is retracted within the storage compartment in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an automatic toilet bowl covering device that effectively prevents the dispersion of airborne waste particles during flushing. There also exists a long-felt need in the art for an automatic toilet bowl covering device that minimizes water splashes and prevents contamination of surrounding surfaces. Moreover, there exists a long-felt need in the art for an automatic toilet bowl covering device that automates the process of deploying and retracting a protective cover, ensuring consistent hygiene and ease of use.

The present invention, in one exemplary embodiment, is comprised of an automatic toilet bowl covering device. The automatic toilet bowl covering device is a motorized system designed to deploy a flexible or hard cover over the toilet bowl opening, limiting the dispersion of airborne waste particles during flushing.

The flexible or hard cover is comprised of an outer layer with hydrophobic and oleophobic coatings to repel liquids and particles, a middle layer of reinforced mesh for tear resistance and pliability, and an inner layer with an antimicrobial coating to inhibit bacterial growth and minimize odor. Accordion-style folds facilitate smooth retraction into a storage compartment, while elastic edging ensures a secure seal around the bowl opening.

The storage compartment, located at the rear of the toilet seat, is constructed from durable materials and incorporates a UV-C light for sterilizing the cover between uses. The UV-C light, powered by a battery with a status indicator, neutralizes pathogens to ensure hygienic readiness.

A motorized mechanism controls the deployment and retraction of the cover, using a brushless DC motor connected to guide roller tracks positioned around the toilet bowl perimeter. The cover retracts compactly into the compartment, where the UV-C light continues to sterilize. Automatic operation is facilitated by sensors: a weight sensor detects when the user rises, motion sensors monitor the lid position, and a flush detection sensor triggers retraction after a delay for effective particle containment.

The device improves bathroom hygiene by containing waste particles, reducing contamination, and minimizing the need for manual cleaning. The durable, multi-layered cover and integrated UV-C sterilization system provide long-term reliability and convenience.

Accordingly, the automatic toilet bowl covering device is particularly beneficial as it deploys a flexible or hard, multi-layered cover over the toilet bowl opening during flushing. The cover prevents the escape of waste particles and splashes, protecting surrounding surfaces and maintaining hygiene. The integration of features such as a motorized deployment mechanism, weight sensors, and UV-C light sterilization ensures automated operation and consistent cover sanitization. By combining durability, ease of maintenance, and effective containment, the device offers a practical and innovative solution for enhancing bathroom cleanliness and reducing contamination risks.

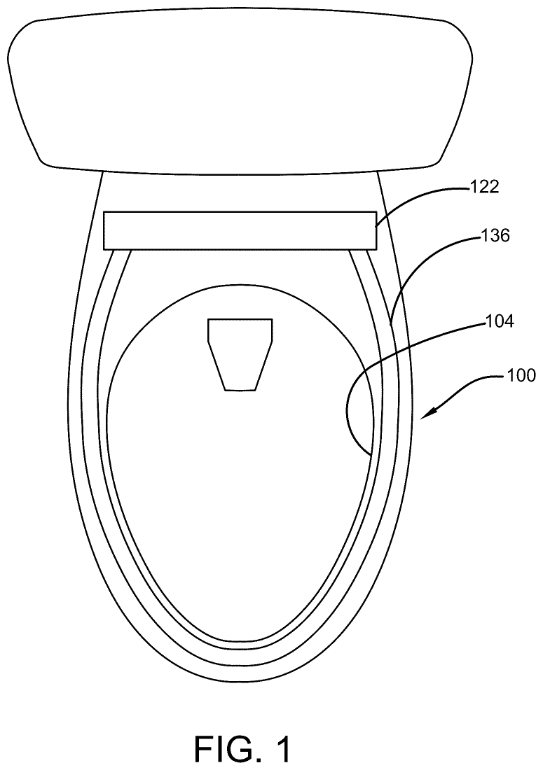

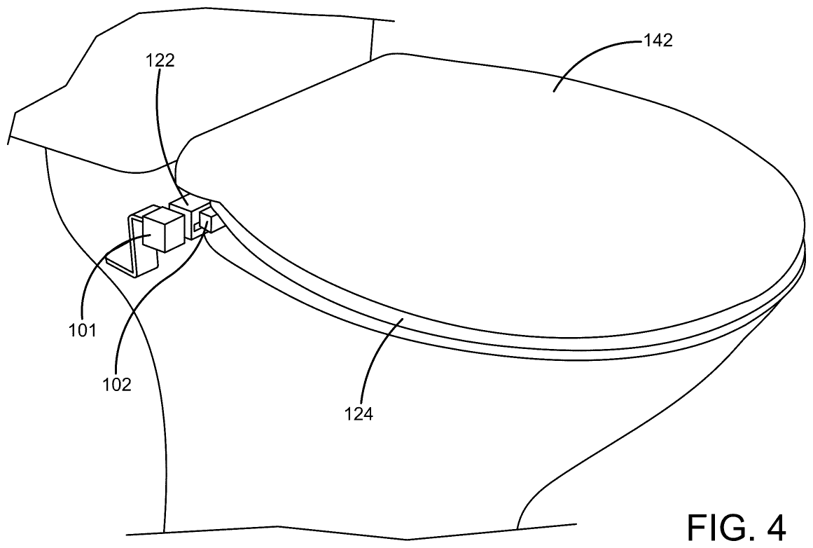

Referring initially to the drawings, FIG. 1 illustrates a top view of one potential embodiment of an automatic toilet bowl covering device 100 of the present invention while attached to a toilet and wherein the cover 102 is retracted within the storage compartment 122 in accordance with the disclosed architecture. The automatic toilet bowl covering device 100 is a compact, motorized device designed to deploy a flexible or hard cover 102 across the toilet bowl opening 104, effectively preventing the dispersion of airborne waste particles during the flushing process. The device 100 attaches to a toilet via at least one fastener 101, as seen in FIG. 4, such as but not limited to a clip or a clamp.

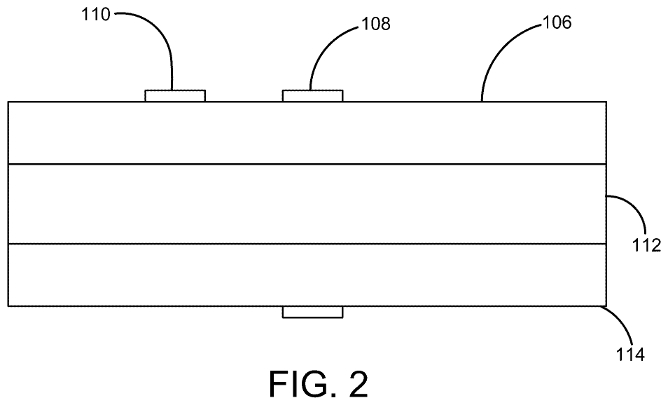

The flexible or hard cover 102 is constructed with three layers, as seen in FIG. 2, each designed for specific purposes. The outer layer 106 is treated with a hydrophobic coating 108 and an oleophobic coating 110. These coatings 108,110 repel liquids and particles, ensuring that the cover 102 remains clean and easy to maintain even after exposure to splashes or contaminants. The middle layer 112 is made from a reinforced, flexible mesh material, providing tear resistance while maintaining the pliability required for smooth folding and deployment. The inner layer 114 is comprised of an antimicrobial coating 116 that inhibits bacterial growth and minimizes odor retention.

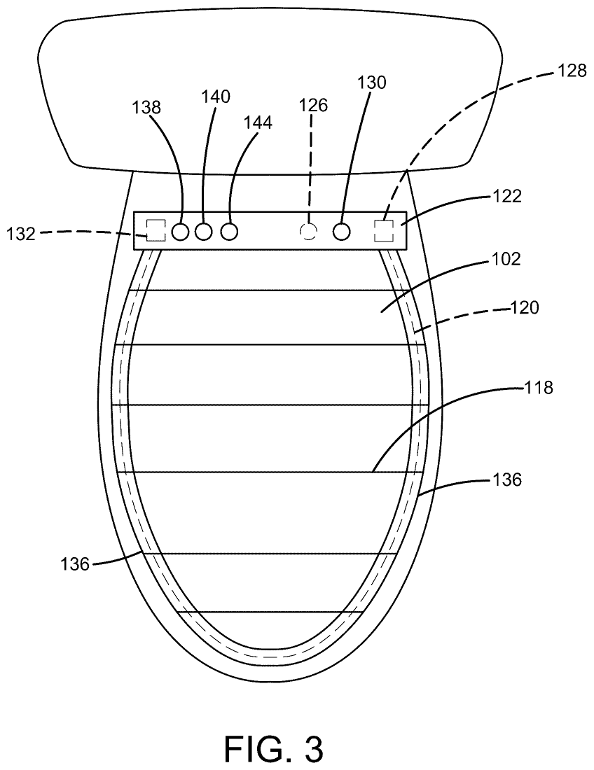

The cover 102 features at least one accordion-style fold 118, which allows for smooth, compact retraction into the storage compartment 122 and facilitates deployment over the toilet bowl opening 104, as seen in FIG. 3. Elastic edging 120 is integrated along the perimeter of the cover 102 to ensure a tight, secure seal around the bowl opening 104, effectively containing particles and preventing their dispersion during the flushing process. When the cover 102 is not in use, storage occurs within a compartment 122 discreetly positioned at the rear of the toilet seat 124. The compartment 122 is constructed from durable materials, such as ABS plastic or stainless steel, which resist wear and corrosion. Inside the compartment 122, a UV-C light 126 sterilizes the cover 102 after each use. The sterilization process neutralizes bacteria, viruses, and other pathogens, ensuring the cover 102 remains hygienic and ready for subsequent use. The UV-C light 126 is powered by at least one battery 128, with the battery status monitored through an indicator 130, such as an LED display or color-coded signal. The indicator 130 alerts users when the battery 128 requires recharging or replacement.

The deployment and retraction of the cover 102 are controlled by a motorized mechanism 132. The mechanism 132 is preferably comprised of a brushless DC motor. The motor 132 connects to a series of guide roller tracks 136 that line the perimeter of the toilet bowl opening 104 underneath a toilet seat. During the retraction process, the motor 132 operates in reverse, pulling the cover 102 back into the compartment 122. The pre-formed accordion-style folds 118 ensure the cover 102 compacts neatly into the compartment 122 without tangling. While the cover 102 retracts, the UV-C light 126 continues to sterilize the surface to maintain hygiene for future use.

The automatic nature of the device 100 eliminates the need for manual intervention. More specifically, a weight sensor 138 detects when a user rises from the toilet seat 124, triggering the deployment process for the cover 102. Additional motion sensors 140 monitor the position of the toilet lid 142 to ensure proper activation of the system. A flush detection sensor 144, such as a water flow or vibration sensor, identifies when flushing occurs and signals the device 100 to begin the retraction process. The device 100 incorporates a brief delay after flushing to allow sufficient time for particle containment before the cover 102 retracts. The motor 132 then engages to smoothly deploy or retract the cover 102 along the guide tracks 136, maintaining consistent performance throughout the operation.

The automatic toilet bowl covering device 100 offers significant advantages over traditional toilet designs. By effectively containing airborne waste particles, the device enhances bathroom hygiene and prevents contamination of surrounding surfaces. The multi-layer construction of the cover 102 ensures durability, tear resistance, and long-term reliability, while the integrated UV-C sterilization system 126 eliminates the need for manual cleaning.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “automatic toilet bowl covering device” and “device” are interchangeable and refer to the automatic toilet bowl covering device 100 of the present invention.

Notwithstanding the forgoing, the automatic toilet bowl covering device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the automatic toilet bowl covering device 100 as shown in the FIGS. are for illustrative purposes only, and that many other sizes and shapes of the automatic toilet bowl covering device 100 are well within the scope of the present disclosure. Although the dimensions of the automatic toilet bowl covering device 100 are important design parameters for user convenience, the automatic toilet bowl covering device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An automatic toilet bowl covering device comprising:
a cover configured to deploy across a toilet bowl opening and retract into a storage compartment, the storage compartment positioned at a rear of a toilet bowl;
a guide roller track;
a motorized mechanism operatively connected to the cover, the motorized mechanism configured to facilitate deployment and retraction of the cover from the storage compartment; and
a fastener configured to attach the automatic toilet bowl covering device to a toilet.

2. The automatic toilet bowl covering device of claim 1, wherein the cover is comprised of an accordion-style fold.

3. The automatic toilet bowl covering device of claim 1, wherein the cover is comprised of an elastic edge.

4. The automatic toilet bowl covering device of claim 1, wherein the cover is comprised of a hydrophobic coating.

5. The automatic toilet bowl covering device of claim 1, wherein the cover is comprised of an oleophobic coating.

6. An automatic toilet bowl covering device comprising:
a cover configured to deploy across a toilet bowl opening and retract into a storage compartment, the storage compartment positioned at a rear of a toilet bowl;
a guide roller track;
a UV-C light positioned within the storage compartment configured to sterilize the cover during retraction
a motorized mechanism operatively connected to the cover, the motorized mechanism configured to facilitate deployment and retraction of the cover from the storage compartment; and
a fastener configured to attach the automatic toilet bowl covering device to a toilet.

7. The automatic toilet bowl covering device of claim 6 further comprised of a battery.

8. The automatic toilet bowl covering device of claim 7 further comprised of a status indicator that indicates a charge level of the battery.

9. The automatic toilet bowl covering device of claim 6, wherein the cover is comprised of a middle layer comprised of a flexible mesh.

10. The automatic toilet bowl covering device of claim 6 further comprised of a fastener.

11. The automatic toilet bowl covering device of claim 10, wherein the fastener is comprised of a clip.

12. An automatic toilet bowl covering device comprising:
a cover configured to deploy across a toilet bowl opening and retract into a storage compartment, the storage compartment positioned at a rear of a toilet bowl;
a guide roller track;
a UV-C light positioned within the storage compartment configured to sterilize the cover during retraction;
a sensor;
a motorized mechanism operatively connected to the cover, the motorized mechanism configured to facilitate deployment and retraction of the cover from the storage compartment; and
a fastener configured to attach the automatic toilet bowl covering device to a toilet.

13. The automatic toilet bowl covering device of claim 12, wherein the sensor is comprised of a weight sensor.

14. The automatic toilet bowl covering device of claim 12, wherein the sensor is comprised of a flush sensor.

15. The automatic toilet bowl covering device of claim 12, wherein the sensor is comprised of a motion sensor.

16. The automatic toilet bowl covering device of claim 12, wherein the sensor controls the deployment and the retraction of the cover from the storage compartment.

17. The automatic toilet bowl covering device of claim 12 further comprised of a fastener.

18. The automatic toilet bowl covering device of claim 17 further comprised of a clamp.

19. The automatic toilet bowl covering device of claim 18, wherein the cover is comprised of a flexible material.

20. The automatic toilet bowl covering device of claim 12, wherein the cover is comprised of a hard material and an elastic edge.
